(19) United States
(12) Patent Application Publication
(10) Pub. No.: US 2023/0372226 A1
(43) Pub. Date: Nov. 23, 2023

(54) HAIR CONDITIONER COMPOSITIONS CONTAINING NON-SILICONE CONDITIONING AGENTS

(71) Applicant: The Procter & Gamble Company, Cincinnati, OH (US)

(72) Inventors: Jean Jianqun Zhao, Cincinnati, OH (US); Jennifer Mary Marsh, Deerfield Township, OH (US); Michelle Ramos, Loveland, OH (US); David Salloum Salloum, West Chester, OH (US)

(21) Appl. No.: 18/318,805

(22) Filed: May 17, 2023

Related U.S. Application Data
(60) Provisional application No. 63/342,638, filed on May 17, 2022.

(51) Int. Cl. A61K 8/85; A61K 8/37; A61K 8/41; A61K 8/04; A61K 8/92; A61Q 5/12

(52) U.S. Cl. CPC A61K 8/85; A61K 8/37; A61K 8/41; A61K 8/042; A61K 8/922; A61Q 5/12; A61K 2800/5426; A61K 2800/34

(57) ABSTRACT

A hair conditioner composition comprising: (a) a L-basal lamellar gel network; (b) from about 0.01 wt % to about 5 wt % of a dicarboxylic acid amine salt; (c) from about 0.01 wt % to about 5 wt % of a diester; (d) from about 0.01 wt % to about 5 wt % of a glycerin ester copolymer.

### FIELD OF THE INVENTION

The present invention relates to hair conditioner compositions, more particularly to hair conditioner compositions comprising non-silicone conditioning agents.

### BACKGROUND OF THE INVENTION

A variety of approaches have been developed to condition the hair. These approaches range from post-shampoo application of hair conditioners such as leave-on and rinse-off products, to hair conditioning shampoos that attempt to both clean and condition the hair from a single product.

Although some consumers prefer the ease and convenience of a shampoo which includes conditioners, a substantial proportion of consumers prefer the more conventional conditioner formulations which are applied to the hair as a separate step from shampooing, usually after shampooing. Conditioning formulations can be in the form of rinse-off products or leave-on products, and can be in the form of an emulsion, cream, gel, spray, or mousse. Such consumers who prefer the conventional conditioner formulations value the relatively higher conditioning effect, or the convenience of changing the amount of conditioning depending on the condition of hair or amount of hair.

Silicone fluids are widely used in hair conditioners to provide a variety of hair benefits such as a reduction of combing force, improved slip feel, increased shine of hair, prevention of frizz, and retention of hair styles. Most frequently-used silicone fluids in hair conditioners include dimethicones, cyclomethicones, phenyl trimethicones, dimethiconols, aminosilicones, amodimethicones, pendant quaternary ammonium silicones, terminal quaternary ammonium silicones, amino polyalkylene oxide silicones, quaternary ammonium polyalkylene oxide silicones, and amino morpholino silicones. However, silicone is not easily washed off during shampooing, and, over time, silicone can build up on the hair surface, making the hair heavy and weighing it down. In addition, most of the silicones are not readily biodegradable and do not meet environmental sustainability requirements. Thus, some consumers would prefer to not have silicones in hair care products, and there is a trend towards beauty products being substantially free of silicones.

Natural oils and waxes have been formulated into hair conditioners to replace silicone for hair conditioning. They are typically botanical triglyceride oils and waxes such as coconut oil, shea butter, cocoa butter, pequi oil, argan oil, almond oil, apricot oil, rice bran oil, safflower oil, sunflower oil, hemp seed oil, avocado oil, grapeseed oil, evening primrose oil, camelia oil, moringa oil, meadowfoam oil, crambe oil, jojoba oil, castor oil, cottonseed oil, soybean oil, rapeseed oil, canola oil, candelilla wax, rice bran wax, sunflower wax, beeswax, bayberry wax, orange wax, and carnauba wax. The key consumer benefits of using natural oils in hair conditioners are hair moisturization and scalp health. However, there can be drawbacks, such as a draggy feel during the wet rinse, oils balling up on the dry hair surface resulting in an oily and greasy feel, and difficulty in creating and maintaining hair styles.

Thus, there is a need for hair conditioners comprising non-silicone hair conditioning materials that are still able to provide to consumers the advantages and properties of conditioners with silicones.

### SUMMARY OF THE INVENTION

A hair conditioner composition comprising: (a) a L-basal lamellar gel network; (b) from about 0.01 wt % to about 5 wt % of a dicarboxylic acid amine salt; (c) from about 0.01 wt % to about 5 wt % of a diester; (d) from about 0.01 wt % to about 5 wt % of a glycerin ester copolymer.

### DETAILED DESCRIPTION OF THE INVENTION

While the specification concludes with claims particularly pointing out and distinctly claiming the invention, it is believed that the present invention will be better understood from the following description.

Hair conditioner compositions of the present invention may comprise: (a) a L-basal lamellar gel network; (b) from about 0.01 wt % to about 5 wt % of a dicarboxylic acid amine salt; (c) from about 0.01 wt % to about 5 wt % of a diester; (d) from about 0.01 wt % to about 5 wt % of a glycerin ester copolymer; wherein the L-basal lamellar gel network composition comprises (i) an aqueous carrier; (ii) from about 0.1 wt % to about 20 wt % of a cationic surfactant; (iii) from about 0.1 wt % to about 20 wt % of a fatty alcohol; wherein the L-basal lamellar gel network comprises d-spacing of from about 5 nm to about 50 nm, as measured according to the d-spacing (L -basal spacing) of Lamella Gel Network Test Method; and wherein the composition has a shear stress from about 40 Pa to about 800 Pa @ 950 1/s.

Hair conditioners are used to improve the feel, appearance, and manageability of the hair. Hair conditioning compositions generally comprise a L-basal lamellar gel network structure that is formed by the process of making (e.g., heating, emulsifying, and cooling) of compositions comprising (i) cationic surfactant(s), (ii) high melting point fatty compound(s) having a melting point of greater than 25° C. and in some examples from 40° C. to 85° C., and (iii) an aqueous carrier. The L-basal lamellar gel network structure provides: (a) consumer-preferred cosmetic appearance of creamy in-hand texture, slippery feel, and richness when spreading, (b) wet conditioning benefits, including improved wet detangling of the hair and slippery rinse feel, (c) dry hair protection benefits such as the repair of damaged hair and anti-statics, and (d) structure robustness to suspend and deliver hair conditioning active ingredients such as silicones, oils and particles.

To deliver consumer benefits and to have the structure robustness of hair conditioner compositions, in this invention, the preferred L-basal lamellar gel network structure comprises d-spacing of from about 5 nm to about 50 nm, as measured according to the d-spacing (L -basal spacing) of Lamella Gel Network Test Method. Also, the composition may have a shear stress from about 40 Pa to about 800 Pa @ 950 1/s.

Silicone has been used in hair conditioner compositions to provide hair benefits such as smooth hair feel, hair shine, hair moisturization, damaged hair repair, hair manageability, hair styling, and curl retention. In order to deliver such consumer benefits, the present inventive non-silicone hair conditioning composition may include (a) a dicarboxylic acid amine salt, (b) a diester, (c) and a glycerin ester copolymer.

Dicarboxylic acid amine salt may be used in this inventive composition to increase the deposition of conditioning actives on the hair surface that delivers the benefits of improved hair moisturization, hair softness, anti-statics, and damaged hair repair. The level in the composition may be from about 0.01 wt % to about 5 wt %. Too high a level of the dicarboxylic acid amine salt may result in over-deposition on the hair surface, causing hair to be weighed down with reduced hair volume. It can also generate a greasy and dirty feel.

Diester may be used in this composition to provide lubrication of the conditioner composition, which delivers consumer-preferred hair surface smoothness, increased slippery feel of the hair, and enhanced hair shine. The level in the composition may be from about 0.01 wt % to about 5 wt %. Too high a level of diester may migrate into the preferred L-basal lamellar gel network structure and reduce the product robustness.

Glycerin ester copolymer may be used in this composition to provide thin film formation on the hair surface, which delivers hair surface smoothness, increased hair bounce and flexibility, damaged hair repairs, improved curl retention, and increased hair manageability. The level in the composition may be from about 0.01 wt % to about 5 wt %. Too high a level of glycerin ester copolymer may result in difficulty spreading it on the hair surface and in over-deposition on the hair surface, causing hair weigh-down with reduced hair volume. It can also generate a greasy and dirty feel.

Surprisingly, the inventive hair conditioner compositions comprising: (a) a L-basal lamellar gel network; (b) from about 0.01 wt % to about 5 wt % of a dicarboxylic acid amine salt; (c) from about 0.01 wt % to about 5 wt % of a diester; (d) from about 0.01 wt % to about 5 wt % of a glycerin ester copolymer can provide consumer delighted benefits without using silicone.

Furthermore, natural botanical oils or waxes from plants and/or vegetables have been used as hair conditioning actives to provide hair conditioning benefits. However, the drawbacks are thatthe oils may reduce the slippery wet rinse feel during washing, and may cause non-even spreading on the hair surface with oil droplets balling up on the hair surface, resulting in a greasy feel and weighing the hair down.

Surprisingly, the inventive hair conditioner composition comprising: (a) a L-basal lamellar gel network; (b) from about 0.01 wt % to about 5 wt % of a dicarboxylic acid amine salt; (c) from about 0.01 wt % to about 5 wt % of a diester; (d) from about 0.01 wt % to about 5 wt % of a glycerin ester copolymer; (e) about 0.1 wt % to about 15 wt % of a natural oil or wax can provide consumer delighted benefits without using silicone.

### L-Basal Lamellar Gel Network

The conditioners of the present invention may comprise a L-basal lamellar gel network that can provide conditioning benefits, including improved wet detangling during wash and wet feel of the hair after rinsing of the conditioner. As used herein, the term “gel network” refers to a lamellar or vesicular solid crystalline phase which comprises at least one high melting point fatty compound, such as a fatty alcohol, as specified below, at least one surfactant, in particular a cationic surfactant, as specified below, and water or other suitable solvents. The lamellar or vesicular phase comprises bi-layers made up of a first layer comprising the high melting point fatty compound and the surfactant and alternating with a second layer comprising the water or other suitable solvent. Gel networks, generally, are further described by G. M. Eccleston, “Functions of Mixed Emulsifiers and Emulsifying Waxes in Dermatological Lotions and Creams”, Colloids and Surfaces A: Physiochem. and Eng. Aspects 123-124 (1997) 169-182; and by G. M Eccleston, “The Microstructure of Semisolid Creams”, Pharmacy International, Vol. 7, 63-70 (1986).

A L-basal lamellar gel network can be formed by (a) a cationic surfactant, (b) a high melting point fatty compound, and (c) an aqueous carrier. The L-basal lamellar gel network is suitable for providing various conditioning benefits, such as slippery feel during the application to wet hair and softness and moisturized feel on dry hair.

Alternatively, when the L-basal lamellar gel network is formed, the cationic surfactant and the high melting point fatty compound are contained at a level such that the weight ratio of the cationic surfactant to the high melting point fatty compound is in the range of, alternatively from about 1:1 to about 1:10, alternatively from about 1:1 to about 1:7, alternatively from about 1:1.5 to about 1:7, alternatively from about 1:1.5 to about 1:5, alternatively from about 1:2 to about 1:6, alternatively from about 1:2 to about 1:5, in view of providing improved wet conditioning benefits.

Alternatively, especially when the L-basal lamellar gel network is formed, the composition of the present invention is substantially free of anionic surfactants, in view of stability of the gel network. In the present invention, “the composition being substantially free of anionic surfactants” means that: the composition is free of anionic surfactants; or, if the composition contains anionic surfactants, the level of such anionic surfactants is very low. In the present invention, a total level of such anionic surfactants, if included, may be alternatively 1% or less, alternatively 0.5% or less, alternatively 0.1% or less by weight of the composition. Most alternatively, the total level of such anionic surfactants is 0% by weight of the composition.

Alternatively, when the L-basal lamellar gel network is formed, the L-basal lamellar gel network may comprise d-spacing of from about 5 nm to about 50 nm, alternatively from about 8 nm to about 45 nm, alternatively from about 10 nm to about 40 nm, and alternatively from about 12 nm to about 35 nm, as measured according to the d-spacing (L -basal spacing) of Lamella Gel Network Test Method. The compositions of the present invention may have a shear stress from about 40 Pa to about 800 Pa @ 950 1/s, alternatively from about 50 Pa to about 700 Pa @ 950 1/s, alternatively from about 50 Pa to about 600 Pa @ 950 1/s, and alternatively from about 60 Pa to about 600 Pa @ 950 1/s.

### Cationic Surfactant

The compositions of the present invention can comprise a cationic surfactant. The cationic surfactant can be included in the composition at a level of from about 0.1%, alternatively from about 0.5%, alternatively from about 0.8%, alternatively from about 1.0%, and to about 20%, alternatively to about 15%, alternatively to about 12%, alternatively to about 10%, alternatively to about 8.0%, alternatively to about 6.0% by weight of the composition, in view of providing the benefits of the present invention.

The surfactant can be water-insoluble. In the present invention, “water-insoluble surfactants” means that the surfactants have a solubility in water at 25° C. of alternatively below 0.5 g/100 g (excluding 0.5 g/100 g) water, alternatively 0.3 g/100 g water or less.

Cationic surfactant can be one cationic surfactant or a mixture of two or more cationic surfactants. Alternatively, the cationic surfactant is selected from: a mono-long alkyl amine; a di-long alkyl quaternized ammonium salt; mono-long alkyl cationic neutralized amino acid esters; a combination of a mono-long alkyl amine and a di-long alkyl quaternized ammonium salt; and a combination of a mono-long alkyl amine and a mono-long alkyl cationic neutralized amino acid esters.

### Mono-Long Alkyl Amine

Mono-long alkyl amine can include those having one long alkyl chain of alternatively from 19 to 30 carbon atoms, alternatively from 19 to 24 carbon atoms, alternatively from 20 to 24 carbon atoms, alternatively from 20 to 22 alkyl group. Mono-long alkyl amines can include mono-long alkyl amidoamines. Primary, secondary, and tertiary fatty amines can be used.

Tertiary amido amines having an alkyl group of from about 19 to about 22 carbons. Exemplary tertiary amido amines include: behenamidopropyldimethylamine, behenamidopropyldiethylamine, behenamidoethyldiethylamine, behenamidoethyldimethylamine, brassicamidopropyldimethylamine, brassicamidopropyldiethylamine, brassicamidoethyldiethylamine, brassicamidoethyldimethylamine. Amines in the present invention are disclosed in U.S. Pat. No. 4,275,055, Nachtigal, et al.

In some examples, the conditioner composition can be substantially free of or free of stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, arachidamidopropyldimethylamine, arachidamidopropyldiethylamine, arachidamidoethyldiethylamine, arachidamidoethyldimethylamine, and/or diethylaminoethylstearamide.

These amines are used in combination with acids such as □-glutamic acid, lactic acid, hydrochloric acid, malic acid, succinic acid, acetic acid, fumaric acid, tartaric acid, citric acid, □-glutamic hydrochloride, maleic acid, and mixtures thereof; alternatively lactic acid, citric acid, at a molar ratio of the amine to the acid of from about 1:0.3 to about 1:2, alternatively from about 1:0.4 to about 1:1. The conditioner composition can contain from about 0.25 wt % to about 6 wt % acid, alternatively from about 0.4 wt % to about 5 wt % acid, from about 0.5 wt % to about 4 wt % acid, and alternatively from about 0.6 wt % to about 3 wt % acid.

In some examples, the conditioner composition can be free of mono long alkyl quaternized ammonium salts.

### Mono-Long Alkyl Quaternized Ammonium Salt

The mono-long alkyl quaternized ammonium salts useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxy alkyl, aryl or alkylaryl group having up to about 4 carbon atoms.

Mono-long alkyl quaternized ammonium salts useful herein are those having the formula (I):

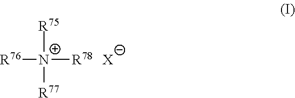

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkykl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt, stearyl trimethyl ammonium salt; cetyl trimethyl ammonium salt; and hydrogenated tallow alkyl trimethyl ammonium salt.

### Di-Long Alkyl Quaternized Ammonium Salts

When used, di-long alkyl quaternized ammonium salts are alternatively combined with a mono-long alkyl quaternized ammonium salt and/or mono-long alkyl amine salt, at the weight ratio of from 1:1 to 1:5, alternatively from 1:1.2 to 1:5, alternatively from 1:1.5 to 1:4, in view of stability in rheology and conditioning benefits.

Di-long alkyl quaternized ammonium salts can have two long alkyl chains of from 12 to 30 carbon atoms, alternatively from 16 to 24 carbon atoms, alternatively from 18 to 22 carbon atoms.

Such di-long alkyl quaternized ammonium salts can have the formula (II):

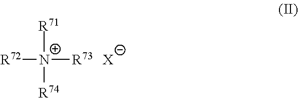

wherein two of R⁷¹, R⁷², R⁷³ and R⁷⁴ are selected from an aliphatic group of from 12 to 30 carbon atoms, alternatively from 16 to 24 carbon atoms, alternatively from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, alternatively from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Alternatively, two of R⁷¹, R⁷², R⁷³ and R⁷⁴ are selected from an alkyl group of from 12 to 30 carbon atoms, alternatively from 16 to 24 carbon atoms, alternatively from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Di-long alkyl cationic surfactants can include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### Alkyl Cationic Neutralized Amino Acid Esters

A neutralized amino acid ester that is a reaction product of a neutral amino acid having a non-polar side chain with a long chain fatty alcohol and is represented by formula (III):

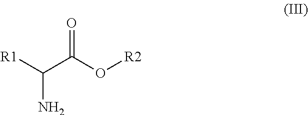

wherein R¹ is a linear or branched alkyl group; R² is a linear or branched carbon chain; and the amine group of the amino acid is neutralized with an acid. The present invention may comprise such materials, including those shown in U.S. Pat. Nos. 8,287,844 B2; 8,105,569 B2; and 11,207,249 B2, which are herein incorporated by reference.

An exemplary preferred neutralized amino acid ester may be Brassicyl L isoleeacine esylate (BLIE) or leucine isostearyl ester esylate (LIEE). Brassicyl L-isoleucine esylate (BLIE) may be derived from the esterification of Brassica alcohol with L-isoleucine esylate. L isoleeacine esylate may be prepared by reacting the amine group on isoleucine with ethanesulfonic acid. Brassica alcohol is a fatty alcohol that is derived from the splitting of high erucic acid rapeseed oil obtained from the Brassica genus of plants followed by hydrogenation. Brassica alcohol consists predominantly of stearyl (C₁₈), arachidyl (C₂₀) and behenyl (C₂₂) alcohols with minor quantities of lower and higher alkyl chain length alcohols. In some embodiments, compositions of the present invention may comprise a neutralized amino acid ester chosen from LIEE, BLIE or a combination thereof. Some enibodiments may comprise brassicyl valinate esylate (BVE).

### High Melting Point Fatty Compound

The composition of the present invention may comprise a high melting point fatty compound. The high melting point fatty compound can be included in the composition at a level of from about 1.0%, alternatively from about 1.5%, alternatively from about 2.0%, alternatively from about 2.5%, even alternatively from about 3%, and to about 30%, alternatively to about 15%, alternatively to about 8.0%, alternatively to about 7% by weight of the composition, in view of providing the benefits of the present invention.

The high melting point fatty compound can have a melting point of 25° C. or higher, alternatively 40° C. or higher, alternatively 45° C. or higher, alternatively 47° C. or higher, alternatively 49° C. or higher, in view of stability of the emulsion especially the gel network. Alternatively, such melting point is up to about 90° C., alternatively up to about 80° C., alternatively up to about 75° C., even alternatively up to about 71° C., in view of easier manufacturing and easier emulsification. In the present invention, the high melting point fatty compound can be used as a single compound or as a blend or mixture of at least two high melting point fatty compounds. When used as such blend or mixture, the above melting point means the melting point of the blend or mixture.

The high melting point fatty compound can be selected from the group consisting of fatty alcohols, fatty acids, and mixtures thereof. Further, it is understood by the artisan that, depending on the number and position of double bonds, and length and position of the branches, certain compounds having certain required carbon atoms may have a melting point of less than the above preferred in the present invention. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are alternatively used in the composition of the present invention. The fatty alcohols can have from about 14 to about 30 carbon atoms, alternatively from about 16 to about 22 carbon atoms. These fatty alcohols are saturated and can be straight or branched chain alcohols.

Fatty alcohols can include, for example, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl, brassica or behenyl group.

The fatty alcohol can be a mixture of cetyl alcohol and stearyl alcohol.

Generally, in the mixture, the weight ratio of cetyl alcohol to stearyl alcohol is alternatively from about 1:9 to 9:1, alternatively from about 1:4 to about 4:1, alternatively from about 1:2.3 to about 1.5:1, alternatively from about 1:2 to about 1.2:1, alternatively from about 1:1.2 to about 1.2:1.

When using higher level of total cationic surfactant and high melting point fatty compounds, the mixture has the weight ratio of cetyl alcohol to stearyl alcohol of alternatively from about 1:1 to about 4:1, alternatively from about 1:1 to about 2:1, alternatively from about 1.2:1 to about 2:1, in view of avoiding too thick for ease of spreadability. It may also provide more conditioning on damaged part of the hair.

### Aqueous Carrier

The composition of the present invention can include an aqueous carrier. The level and species of the carrier can be selected according to the compatibility with other components, and other desired characteristics of the product.

The carrier can include water and water solutions of lower alkyl alcohols. The lower alkyl alcohols can be monohydric alcohols having 1 to 6 carbons, alternatively ethanol and isopropanol.

Alternatively, the aqueous carrier is substantially water. Deionized water is alternatively used. Water from natural sources including mineral cations can also be used, depending on the desired characteristic of the product. Generally, the compositions of the present invention comprise from about 40% to about 99%, alternatively from about 50% to about 95%, and alternatively from about 70% to about 93%, and alternatively from about 80% to about 92% water.

### Dicarboxylic Acid Amine Salt

The composition of the present invention may comprise a dicarboxylic acid amine salt. The dicarboxylic acid amine salt can be included in the composition at a level of from about 0.01 wt %, alternatively from about 0.05 wt %, alternatively from about 0.1 wt %, alternatively from about 0.15 wt %, even alternatively from about 0.2 wt %, and to about 5 wt %, alternatively to about 4 wt %, alternatively to about 3 wt %, alternatively to about 2 wt % %, alternatively to about 1 wt % of the composition, in view of providing the benefits of the present invention.

The dicarboxylic acid amine salts useful herein may be those having the formula (IV):

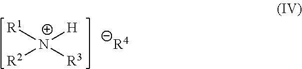

wherein R¹ is a C₈ to C₄₀ (preferably a C₁₂ to C₃₆) straight or branch-chained, saturated or unsaturated hydrocarbon group (preferably a saturated or alkyl group) or a fatty amido group according to the formula (V):

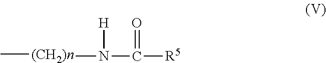

wherein R⁵ is a C₈ to C₄₀ (preferably a C₁₂ to C₃₆, even more preferably a C₁₂ to C₂₂) straight or branch-chained, saturated or unsaturated hydrocarbon group (preferably a saturated or alkyl group), n is from 1 to 5, preferably 2 or 3, more preferably 3 (propylene group);
R² and R³ are methyl, ethyl or propyl groups, preferably methyl groups;
wherein R⁴ is a C₂ to C₄₀ (preferably a C₃ to C₃₆) diacid dianionic (both carboxylic acid residues are ionized as carboxylate anions) residue (obtained from neutralization of a dibasic acid) according to the formula (VI):

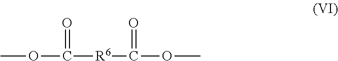

wherein the R⁶ is a C₂ to C₄₀ (preferably a C₃ to C₃₆) straight or branch-chained, saturated or unsaturated hydrocarbon group;
wherein the formula (IV) is a salt of a dicarboxylic acid and an amine. wherein the dicarboxylic acid is selected from C3 to C36 alkyl dicarboxylic acid, dilinoleic acid, C36 aliphatic alkyl dicarboxylic acid, C36 monocycloaliphatic alkyl dicarboxylic acid, and mixtures thereof; preferably, a dimer dilinoleic acid; and wherein the amine is selected from: mono-long alkyl amines; mono-long alkyl dimethylamine, mono-long alkyl amidoamines, mono-long alkyl amidopropyl dimethylamines, lauryldimethylamine, hexadecyldimethylamine, linoleamidopropyldimethylamine, behenamidopropyldimethylamine, behenamidopropyldiethylamine, behenamidoethyldiethylamine, behenamidoethyldimethylamine, brassicamidopropyldimethylamine, brassicamidopropyldiethylamine, brassicamidoethyldiethylamine, brassicamidoethyldimethylamine amines, stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, arachidamidopropyldimethylamine, arachidamidopropyldiethylamine, arachidamidoethyldiethylamine, arachidamidoethyldimethylamine, and/or diethylaminoethylstearamide, and mixtures thereof.

The present invention may comprise such materials, including those dicarboxylic acid amine salts shown in U.S. Pat. Nos. 4,548,810A and 6,723,310B2, which are herein incorporated by reference.

The preferred dicarboxylic acid amine salt may be linoleamidopropyldimethylamine dimer dilinoleate which is available from Alzo International Inc (Sayreville, N.J. USA) under the trade name of Necon LO-80, lauryldimethylamine dimer dilinoleate which is available from Alzo International Inc (Sayreville, N.J. USA) under the trade name of Necon DLD, behenamidopropyldimethylamine dimer dilinoleate which is available from Alto International Inc (Sayreville, N.J. USA) under the trade name of Necon BD, and the mixture thereof.

### Diester

The composition of the present invention may comprise a diester. The diester can be included in the composition at a level of from about 0.01 wt %, alternatively from about 0.03 wt %, alternatively from about 0.05 wt %, even alternatively from about 0.1 wt %, and to about 5 wt %, alternatively to about 4 wt %, alternatively to about 3 wt %, alternatively to about 2 wt % %, alternatively to about 1 wt %, even alternatively to about 0.5 wt % % of the composition, in view of providing the benefits of the present invention.

The diesters useful herein are those having the formula (VII):

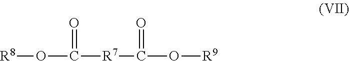

wherein R⁷ is a C₁ to C₄₀ (preferably a C₁ to C₃₆, preferably a C1 to C22, preferably a C1 to C18, preferably a C1 to C16, preferably a C1 to C14, preferably a C1 to C12, preferably a C2 to C12, preferably a C1 to C10, preferably a C2 to C10) straight, cyclic, or branch chained, saturated or unsaturated hydrocarbon group; wherein R⁸ and R⁹ are C₁ to C₄₀ (preferably C₁ to C₃₆, preferably C1 to C22, preferably C1 to C18, preferably C1 to C16, preferably C2 to C16, preferably C4 to C16, preferably C2 to C12) straight, cyclic, or branch chained, saturated or unsaturated hydrocarbon group.

The compositions may comprise a diester selected from the group consisting of diheptyl succinate, dipenyl succinate, didecyl succinate, dicapryl succinate, diheptyl suberate, dipenyl suberate, didecyl suberate, diheptyl sebacate, dipenyl sebacate, didecyl sebacate, diheptyl oxalate, dipenyl oxalate, didecyl oxalate, dioctyl adipate, ditetradecyl sebacate, bis(2thyl-1-hexyl) adipate, and mixtures thereof, wherein the viscosity of the diester is less than about 100 cps, using the cSt viscosity method described herein.

### Glycerin Ester Copolymer

The composition of the present invention may comprise a glycerin ester copolymer. The glycerin ester copolymer can be included in the composition at a level of from about 0.01 wt %, alternatively from about 0.03 wt %, alternatively from about 0.05 wt %, even alternatively from about 0.1 wt %, and to about 5 wt %, alternatively to about 4 wt %, alternatively to about 3 wt %, alternatively to about 2 wt % %, alternatively to about 1 wt %, even alternatively to about 0.5 wt % % of the composition, in view of providing the benefits of the present invention.

The glycerin ester copolymer useful herein is a reaction product of:
(i) at least one polyfunctional alcohol (preferably glycerin),
(ii) at least one polyfunctional carboxylic acid, and
(iii) at least one monofunctional carboxylic acid;
   wherein the polyfunctional alcohol comprises about two to about ten carbon atoms (preferably glycerin); the polyfunctional carboxylic acid comprises one to about thirty-six carbon atoms (preferably sebacic acid); the monofunctional carboxylic acid comprises four to about twenty-four carbon atoms (preferably caprylic acid); and the polyol polyester polymer has a dynamic viscosity at 25° C. of about 200 to about 5000 centipoise and a hydroxyl value of about 40 to about 300 mg KOH/g.

Herein the viscosity for the glycerin ester copolymer was determined using ASTM D-2270, and the hydroxyl number was determined using a modified version of AOCS (American Oil Chemists Society, Champaign, Ill., United States of America), official method number Cd-13-60.

The glycerin ester copolymers in this invention include those complex polyol polyester polymers shown in U.S. Pat. No. 7,317,068 B2, which are herein incorporated by reference.

The preferred glycerin ester copolymer may be capryloyl glycerin/sebacic acid copolymer which is a reaction product of glycerin, sebacic acid and caprylic acid and is available from Inolex (Philadelphia, Pa. USA) under the trade name of Vellaplex™ MB, Lexfilm™ Sun Natural MB, Lipfeel™ Natural MB, Lexfeel™ N5 MB, Lexfeel™ N20 MB, Lexfeel™ N50 MB, Lexfeel™ N100 MB, Lexfeel™ N200 MB, Lexfeel™ N350 MB, and the mixture thereof.

### Botanical Oil or Wax

The composition of the present invention may further comprise a botanical oil or wax.

The botanical oil or wax is selected from the group consisting of natural oils from plants and/or vegetables, coconut oil, corn oil, cottonseed oil, canola oil, olive oil, palm oil, peanut oil, safflower oil, sesame oil, soybean oil, sunflower oil, jojoba oil, shea butter, cocoa butter, pequi oil, argan oil, almond oil, apricot oil, rice bran oil, safflower oil, hemp seed oil, avocado oil, grapeseed oil, evening primrose oil, camelia oil, moringa oil, meadowfoam oil, crambe oil, castor oil, candelilla wax, rice bran wax, sunflower wax, beeswax, bayberry wax, orange wax, carnauba wax, and mixtures thereof.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, alternatively up to about 5% by weight of the composition.

A wide variety of other additional components can be formulated into the present compositions. These include: other conditioning agents such as aloe vera gel; aloe barbadensis leaf juice; ecklonia radiata extract; natural oils and waxes with shea butter, safflower oil, cocoa butter, orange peel wax, olive oil, macadamia seed oil, oenothera biennis oil, crambe abyssinica see oil, argon oil, camelina oil, sunflower oil, almond oil, argania spinosa kernel oil, grape see oil, jojoba oil, coconut oil, meadowfoam seed oil, neem oil, linseed oil, castor oil, soybean oil, sesame oil, beeswax, sunflower wax, candelilla wax, rice bran wax, carnauba wax, bayberry wax and soy wax; essential oils such as lime peel oil, lavender oil, peppermint oil, cedarwood oil, tea tree oil, ylang-ylang oil and coensage oil which can be used in fragrance; hydrolyzed collagen with tradename Peptein 2000 available from Hormel, vitamin E with tradename Emix-d available from Eisai, panthenol available from Roche, panthenyl ethyl ether available from Roche, hydrolyzed keratin, proteins, plant extracts, and nutrients; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; and ultraviolet and infrared screening and absorbing agents such as octyl salicylate; antioxidants include: rosemary, tocopherol, vitamin E, vitamin A,tea extracts, and hydroxyacetophenone (available as SymSave® H from Symrise®); amino acids include histidine, 1-arginine and others.

The conditioner composition can contain from about 0.2 wt % to about 1.5 wt % preservation system, alternatively from about 0.3 wt % to about 1.25 wt % preservation system, alternatively from about 0.4 wt % to about 1 wt % preservation system, alternatively from 0.5 wt % to about 0.8 wt % preservation system, and alternatively from about 0.6 wt % to about 0.8 wt % preservation system.

The conditioner composition can contain from about 0.05 wt % to about 0.8 wt % of a first preservation agent, such as sodium benzoate, alternatively 0.1 wt % to about 0.5 wt % sodium benzoate, alternatively from about 0.2 wt % to about 0.4 wt % sodium benzoate. The conditioner composition can contain sodium benzoate and can contain less than 2% sodium benzoate, alternatively less than 1.5% sodium benzoate, alternatively less than 1% sodium benzoate, alternatively less than 0.8% sodium benzoate, alternatively less than 0.6 wt % sodium benzoate, and alternatively less than 0.5% sodium benzoate.

The preservation system can contain from about 20% to about 50% sodium benzoate, by weight of the preservation system, alternatively from about 25% to about 50% sodium benzoate, by weight of the preservation system, from about 30% to about 50% sodium benzoate, by weight of the preservation system, and from about 30% to about 40% sodium benzoate, by weight of the preservation system.

The conditioner composition can contain from about 0.3 wt % to about 1.5 wt % of a second preservation agent, such as a glycol and/or a glyceryl ester, alternatively from about 0.32 wt % to about 1 wt %, alternatively from about 0.33 wt % to about 0.8 wt %, alternatively from about 0.34 wt % to about 0.6 wt %, alternatively from about 0.35 wt % to about 0.5 wt %, alternatively from about 0.37 wt % to about 0.45 wt %, and alternatively from about 0.38 wt % to about 0.43 wt %. If the conditioner composition contains too much glycol and/or glyceryl esters the gel network structure may be destroyed, and the conditioner will not have consumer acceptable rheology and/or performance.

The preservation system can contain from about 50% to about 80% of the second preservation agent, by weight of the preservation system, alternatively from about 50% to about 75%, by weight of the preservation system, alternatively from about 50% to about 70%, by weight of the preservation system, and alternatively, from about 50% to about 67%, by weight of the preservation system.

The weight ratio of sodium benzoate to the second preservation agent can be from about 1:4 to about 1:1, alternatively from about 1:3 to about 1:1, alternatively from about 1:2 to about 1:1, and from about 1:1.7 to about 1:1.

The conditioner composition can have a shear stress from about 50 Pa to about 600 Pa, alternatively from about 75 Pa to about 575 Pa, alternatively from about 100 Pa to about 565 Pa, alternatively from about 105 Pa to about 550 Pa, alternatively, from about 120 Pa, to about 500 Pa, and alternatively from about 125 Pa to about 450 Pa. The shear stress can be determined using the Shear Stress Test Method, described hereafter.

The conditioner composition can have a pH of less than 5. Alternatively, the conditioner composition can have a pH from about 2.5 to about 5, alternatively from about 3.5 to about 4.5. The pH can be determined using the pH Test Method, described hereafter.

### Perfume

The conditioner compositions disclosed herein can comprise a perfume, which can be referred to as a perfume accord. The perfume can be suitable for application to the hair or skin.

The conditioner composition can contain from about 0.1 wt. % to about 5 wt. % perfume, alternatively from about 0.2 wt. % to about 3 wt. %, alternatively from about 0.3 wt. % to about 4 wt. %, alternatively from about 0.4 wt. % to about 2.5 wt. %, alternatively from about 0.5 wt. % to about 2 wt. %, alternatively from about 0.6 wt. % to about 1.5 wt. %, alternatively from about 0.6 wt. % to about 1.2 wt. %, and alternatively from about 0.7 wt. % to about 1 wt. % based on the total weight of the composition.

A wide variety of chemicals are known for fragrance (i.e., perfume) uses, including materials such as aldehydes, ketones and esters. More commonly, naturally occurring plant and animal oils and exudates comprising complex mixtures of various chemical components are known for use as fragrances. The perfumes can be relatively simple in their compositions, comprising a single chemical, or can comprise highly sophisticated complex mixtures of natural and synthetic chemical components, all chosen to provide any desired odor.

The perfume raw materials of the present compositions can have boiling points (BP) of about 500° C. or lower, alternatively about 400° C. or lower, alternatively about 350° C. or lower. The BP of many perfume raw materials are given in Perfume and Flavor Chemicals (Aroma Chemicals), Steffen Arctander (1969). The C log P value of the perfume raw materials useful herein can be greater than 0.1, alternatively greater than about 0.5, alternatively greater than about 1.0, alternatively greater than about 1.2.

### Soluble Anti-Dandruff Active

The soluble anti-dandruff agent may be one material or a mixture selected from the groups consisting of: azoles, such as climbazole, ketoconazole, itraconazole, econazole, and elubiol; hydroxy pyridones, such as piroctone olamine, ciclopirox, rilopirox, and MEA-Hydroxyoctyloxypyridinone; kerolytic agents, such as salicylic acid and other hydroxy acids; strobilurins such as azoxystrobin and metal chelators such as 1,10-phenanthroline, and hinokitiol. The azole anti-microbials may be an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. The azole anti-microbial agent may be ketoconazole. The sole anti-microbial agent may be ketoconazole.

The soluble anti-dandruff agent may be present in an amount from about 0.1% to 10%, in a further embodiment from about 0.25% to 8%, in yet a further embodiment from about 0.5% to 6%. Alternatively, the soluble anti-dandruff agent may be present in an amount of from about 0.1% to about 2%, alternatively from about 0.15% to about 1.5%, alternatively from about 0.2% to about 1%, alternatively from about 0.2% to about 0.75%, alternatively from about 0.25% to about 0.5%.

### Particulate Anti-Dandruff Agent

The conditioner composition may also contain one or more particulate anti-dandruff agents. A safe and effective amount of anti-dandruff active for control of dandruff of the scalp is used. Particulate antidandruff agents include, for example, sulfur, selenium sulfide, and pyridinethione salts. Preferred are heavy metal salts of 1-hydroxy-2-pyridinethione and selenium disulfide. The particulate anti-dandruff agents are in crystalline form and are insoluble in the compositions. In general, particulate antidandruff agents can be present at levels of about 0.1% to about 5%, preferably from about 0.3% to about 2%, by weight of the composition. The particular amount used is not critical as long as a safe and effective amount is used for controlling dandruff when the composition is used to condition the hair.

### Product Forms

The compositions of the present invention can be in the form of rinse-off products or leave-on products and can be formulated in a wide variety of product forms, including but not limited to creams, gels, emulsions, mousses, and sprays.

The conditioning composition of the present invention is especially suitable for rinse-off hair conditioner. Such compositions are alternatively used by following steps:
(i) after shampooing hair, applying to the hair an effective amount of the conditioning compositions for conditioning the hair; and
(ii) then rinsing the hair.

### Test Methods

### Bacterial Microbial Susceptibility Testing Method

Bacterial microbial susceptibility testing is used to assess the anti-bacterial effectiveness of the preservation system in cosmetic rinse-off conditioner.

A bacterial pool (mixture in equal volumes) of challenge organisms used in the test is comprised of standardized solutions of the bacterial strains Escherichia coli (ATCC# 8739), Staphylococcus aureus (ATCC# 6538), Pseudomonas aeruginosa (ATCC# 9027), Burkholderia cepacia (ATCC#25416), as well as Klebsiella pneumoniae, Enterobacter gergoviae and Serratia marcescens strains isolated from cosmetic products. The bacterial pool is prepared to have a concentration of approximately 6-8 log cfu/ml. To start the test, 0.1 ml of the bacterial pool is added into 10.0 g of a test conditioner. The test conditioner is then incubated for 2 days at 20-25° C. After incubation, a 1.0 g aliquot of product is neutralized using Modified Letheen Broth containing 1.5% polysorbate 80 (commercially available as Tween® 80 from Croda™) and 1% Lecithin to aid in microbial recovery/enumeration. Then, multiple diluted concentrations of this sample are transferred into petri dishes containing Modified Letheen Agar with 1.5% Tween® 80, and the agar plates are incubated at least 2 days at 30-35° C. Bacterial colony forming units (cfus) are then enumerated, and a bacterial log reduction from the starting log cfu/g challenge level is reported.

A 1 log cfu/g reduction equates to ˜ a 90% bacterial reduction. A 2 log cfu/g reduction equates to ˜ a 99% bacterial reduction. A 3 log cfu/g reduction equates to ˜ a 99.9% bacterial reduction. A 4 log cfu/g reduction equates to ˜ a 99.99% bacterial reduction. Greater log cfu/g reduction values indicate greater antimicrobial robustness from the preservation system.

### Fungal Microbial Susceptibility Testing Method

Fungal microbial susceptibility testing is used to assess the anti-fungal effectiveness of the preservation system in cosmetic rinse-off conditioner.

Standardized ATCC strains of the yeast Candida albicans (ATCC# 10231) and mold Aspergillus brasiliensis (frm. niger) (ATCC# 16404) are mixed in 1:1 (v:v) ratio, and this fungal pool is used as inoculum in the test. The concentration of the fungal pool is approximately 6-8 log cfu/ml. To start the test, 0.1 ml of the fungal pool is added into 10.0 g of a testing conditioner. After the inoculated sample is incubated for 2 days at 20-25° C., a 1.0 g aliquot of product is neutralized using Modified Letheen Broth containing 1.5% Tween® 80 and 1% Lecithin to aid in microbial recovery/enumeration. Then, multiple diluted concentrations of this sample are transferred into petri dishes containing Modified Letheen Agar with 1.5% Tween 80, and the agar plates are incubated for at least 5 days at 20-25° C., at which time fungal colony forming units (cfus) are then enumerated, and a fungal log reduction from the starting log cfu/g challenge level is calculated.

A 1 log cfu/g reduction equates to ˜ a 90% fungal reduction. A 2 log cfu/g reduction equates to ˜ a 99% fungal reduction. A 3 log cfu/g reduction equates to ˜ a 99.9% fungal reduction. A 4 log cfu/g reduction equates to ˜ a 99.99% fungal reduction. Greater log cfu/g reduction values indicate greater anti-fungal robustness from the preservation system.

### Differential Scanning Calorimetry

The melt transition behavior and temperature for the gel network may be obtained using differential scanning calorimetry (DSC) according to the following method. Utilizing a TA Instruments Q2000 DSC, approximately 15 mg of the gel network pre-mix or the final conditioner composition containing the gel network is placed into a Tzero aluminum hermetic DSC pan. The sample, along with an empty reference pan is placed into the instrument. The samples are analyzed using the following conditions/temperature program: Nitrogen Purge at a rate of 50.0 mL/min; Equilibrate @ 20.00° C.; Sampling interval 0.10 sec/pt; Equilibrate at 5.00° C.; Isothermal for 1.00 min; Ramp 5.00° C./min to 80.00° C. The resulting DSC data is analyzed using TA Instruments Universal Analysis Software.

The use of DSC to measure the melt transition behavior and temperature for gel networks is further described by T. de Vringer et al., Colloid and Polymer Science, vol. 265, 448-457 (1987); and H. M. Ribeiro et al., Intl. J. of Cosmetic Science, vol. 26, 47-59 (2004).

### pH Method

First, calibrate the Mettler Toledo Seven Compact pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 4 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 7 buffer and press the calibrate button a second time. Wait until the pH icon stops flashing and press the calibrate button a third time. Rinse the electrode with distilled water and carefully wipe the electrode with a scientific cleaning wipe. Then submerse the electrode into the pH 10 buffer and press the calibrate button a third time. Wait until the pH icon stops flashing and press the measure button. Rinse the electrode with distilled water and carefully wipe with a scientific cleaning wipe.

Submerse the electrode into the testing sample and press the read button. Wait until the pH icon stops flashing and record the value.

### Viscosity from Flow Curve Measurement

The viscosities of hair conditioning agents are measured by shear rate sweep condition with a rheometer available from TA Instruments with a mode name of DHR-3. The plate is called Peltier Plate. The temperature of the plate is kept at 25° C. Geometry has 40 mm diameter, cone angle of 2 degree, and gap of 55 μm. Shear rate ramp is between 0.1-1100 1/sec. Viscosities are reported at the shear rate of 2 s⁻¹ and 950 s⁻¹.

### Shear Stress

Shear stress is measured by shear rate sweep condition with a rheometer available from TA Instruments with a mode name of DHR-3. The plate is called Peltier Plate. The temperature of the plate is kept at 25° C. Geometry has 40 mm diameter, cone angle of 2 degree, and gap of 55 μm. Shear rate ramp is between 0.1-1100 1/sec. Shear stress at a high shear rate of 950 s⁻¹ is measured.

### X-Ray Diffraction Method

SAXS (Small Angle X-ray Scattering) is used to confirm the presence of a multi-lamellar phase, and WAXS (Wide Angle X-ray Scattering) is used to differentiate between Lα (liquid) and Lβ (solid) crystalline structures were employed to verify the presence of the characteristic dispersed gel network phase of the personal conditioning compositions

### D-Spacing (Lβ-Basal Spacing) of Lamella Gel Network

Small-angle x-ray scattering (“SAXS”) as used to resolve periodic structures in mesophases is essentially an x-ray diffraction technique. It is used in conjunction with conventional wide-angle x-ray scattering (“WAXS”) to characterize aggregate structures such as micelles, gel networks, lamella, hexagonal and cubic liquid crystals. The different mesophases that show periodic structures can be characterized by the relative positions (d-spacing) of their reflections as derived from the Bragg equation (d=λ/2 Sin θ) where d represents the interplanar spacing, λ the radiation wavelength and θ the scattering (diffraction) angle.

The one-dimensional lamella gel network phase is characterized by the ratio of the interplanar spacings d₁/d₁, d₁/d₂, d₁/d₃, d₁/d₄, d₁/d₅ having the values 1:2:3:4:5 etc. in the SAXS region (long-range order) and one or two invariant reflection(s) in the WAXS region (short-range) centered around 3.5 and 4.5 Å over a broad halo background. Other mesophases (e.g. hexagonal or cubic) will have characteristically different d-spacing ratios.

The SAXS data was collected with a Bruker NanoSTAR small-angle x-ray scattering instrument. The micro-focus Cu x-ray tube was operated at 50 kV, 0.60 mA with 550 um ScanTex Pinholes. The sample to detector distance was 107.39 cm and the detector a Vantec2K 2-dimensional area detector. Samples were sealed in capillaries and analyzed under vacuum with an analysis time of 600 s.

The value of d-spacing ((Lβ-basal spacing) of lamella gel network reported here is obtained with the 1^{st} order of SAXS reflection which is the d₁ spacing.

### WAXS Confirmation (in Combination with SAXS) of Presence of Lβ Gel Network

Wide-angle data (WAXS) was collected on a Stoe STADI-MP diffractometer. The generator was operated at 40 kV/40 mA, powering a copper anode long-fine-focus Cu x-ray tube. The diffractometer incorporates an incident-beam curved germanium-crystal monochromator, standard incident-beam slit system, and Mythen PSD detector. Data were collected in transmission mode over a range of 0° to 50° 2θ with a step size of 3° 2θ and 15 seconds per step.

WAXS Pattern with reflection near 4.2 Å which, in combination with the lamellar reflections seen in the SAXS, is indicative of the presence of Lβ gel network.

### Particle Size: Optical Microscope Image Method

Conditioner compositions are examined under an Olympus BX61 Microscope using an Olympus DP72 camera (ISO 200, Exposure 3 sec) with lamp intensity 10V and air as refractive index (1.003). Microscope pictures were taken with objective lens of both 10× and 50×. Bright Field and Polarized Filter were used to examine the particle sizes of non-silicone hair conditioning agent compositions and gel network formation of conditioner compositions. Olympus cellSense was used as the software for imaging analysis.

### EXAMPLES

The following are non-limiting examples of the conditioner compositions described herein. It will be appreciated that other modifications of the present invention within the skill of those in the art can be undertaken without departing from the spirit and scope of this invention.

All parts, percentages, and ratios herein are by weight unless otherwise specified. Some components may come from suppliers as dilute solutions. The amount stated reflects the weight percent of the added material, unless otherwise specified.

The examples were made as follows. Sodium benzoate and □-glutamic were dissolved in the water. The mixture was heated to 80° C. Then, the cationic surfactant and fatty alcohols (FAOH) were added to the mixture. Next, the mixture was cooled while the cationic surfactant and fatty alcohols continue to dissolve. Then, the additional preservatives were added followed by oils and perfume when the temperature was below 45° C. The composition was cooled to room temperature to make the conditioner composition.

The non-silicone hair conditioning agent compositions were incorporated into the conditioner compositions after the L-basal lamellar gel network formed.

TABLE 1
Compositional Examples

| | Ex. 1 | Ex. 2 | Ex. 3 | Ex. 4 | Ex. 5 |
|---|---|---|---|---|---|
| Sheer stress (Pa) @950 1/s | 430 | 381 | 382 | 355 | 342 |
| DSC peak (melting temp) | 1 peak | 1 peak | 1 peak | 1 peak | 1 peak |
| GN d-spacing (nm) | 21.4 | 21.8 | 21.8 | 21.6 | 21.6 |
| Behenamidopropyl Dimethylamine (BAPDMA)¹ | 3.74 | 3.74 | 3.74 | 3.74 | 3.74 |
| wt % (active) | | | | | |
| L-Glutamic Acid² wt % (active) | 1.35 | 1.35 | 1.35 | 1.35 | 1.35 |
| Cetyl Alcohol (C16 Fatty alcohol)³ | 3.83 | 3.83 | 3.83 | 3.83 | 3.83 |
| wt % (active) | | | | | |
| Stearyl Alcohol (C18 Fatty Alcohol)⁴ | 2.84 | 2.84 | 2.84 | 2.84 | 2.84 |
| wt % (active) | | | | | |
| Sodium Benzoate⁵ wt % (active) | 0.25 | 0.25 | 0.25 | 0.25 | 0.25 |
| Decylene Glycol⁶ wt % (active) | 0.4 | 0.4 | 0.4 | | |
| Caprylyl Glycol⁷ wt % (active) | | | | 0.2 | 0.2 |
| 1,2 Hexandiol⁸ wt % (active) | | | | 0.2 | 0.2 |
| Linoleamidopropyl Dimethylamine Dimer | 1.00 | 0.75 | 0.50 | 1.00 | 0.75 |
| Dilinoleate⁹ wt % (active) | | | | | |
| Diheptyl Succinate¹⁰ wt % (active) | 0.45 | 0.11 | 0.11 | 0.45 | 0.11 |
| Capryloyl Glycerin/Sebacic Acid Copolymer¹¹ | 0.55 | 0.14 | 0.14 | 0.55 | 0.14 |
| wt % (active) | | | | | |
| Distilled Water | Q.S. | Q.S. | Q.S. | Q.S. | Q.S. |
| Adjust pH w Citric acid to pH = 3.5-4.5 | | | | | |

TABLE 2
Comparative Compositional Examples

| | Comp. | Comp. | Comp. | Comp. |
|---|---|---|---|---|
| | Ex. 1 | Ex. 2 | Ex. 3 | Ex. 4 |
| Sheer stress (Pa) @950 1/s | 400 | 324 | 372 | 340 |
| DSC peak (melting temp) | 1 peak | 1 peak | 1 peak | 1 peak |
| GN d-spacing (nm) | 21.6 | 23.4 | 22.0 | 22.4 |
| Behenamidopropyl Dimethylamine (BAPDMA)¹ | 3.74 | 3.74 | 3.74 | 3.74 |
| wt % (active) | | | | |
| L-Glutamic Acid² wt % (active) | 1.35 | 1.35 | 1.35 | 1.35 |
| Cetyl Alcohol (C16 Fatty alcohol)³ | 3.83 | 3.83 | 3.83 | 3.83 |
| wt % (active) | | | | |
| Stearyl Alcohol (C18 Fatty Alcohol)⁴ | 2.84 | 2.84 | 2.84 | 2.84 |
| wt % (active) | | | | |
| Sodium Benzoate⁵ wt % (active) | 0.25 | 0.25 | 0.25 | 0.25 |
| Decylene Glycol⁶ wt % (active) | 0.4 | 0.4 | | |
| Caprylyl Glycol⁷ wt % (active) | | | 0.2 | 0.2 |
| 1,2 Hexandiol⁸ wt % (active) | | | 0.2 | 0.2 |
| Linoleamidopropyl Dimethylamine Dimer | 1.00 | | 1.00 | |
| Dilinoleate⁹ wt % (active) | | | | |
| Diheptyl Succinate¹⁰ wt % (active) | | 0.45 | | 0.45 |
| Capryloyl Glycerin/Sebacic Acid Copolymer¹¹ | | 0.55 | | 0.55 |
| wt % (active) | | | | |
| Distilled Water | Q.S. | Q.S. | Q.S. | Q.S. |
| Adjust pH w Citric acid to pH = 3.5-4.5 | | | | |

TABLE 3
Compositional Examples

| | Ex. 6 | Ex. 7 | Ex. 8 | Ex. 9 | Ex. 10 |
|---|---|---|---|---|---|
| Sheer stress (Pa) @950 1/s | 312 | 302 | 228 | 267 | 263 |
| DSC peak (melting temp) | 1 peak | 1 peak | 1 peak | 1 peak | 1 peak |
| GN d-spacing (nm) | 23.0 | | | | |
| Behenamidopropyl Dimethylamine (BAPDMA)¹ | 3.74 | 3.74 | 3.74 | 3.74 | 3.74 |
| wt % (active) | | | | | |
| L-Glutamic Acid² wt % (active) | 1.35 | 1.35 | 1.35 | 1.35 | 1.35 |
| Cetyl Alcohol (C16 Fatty alcohol)³ | 3.83 | 3.83 | 3.83 | 3.83 | 3.83 |
| wt % (active) | | | | | |
| Stearyl Alcohol (C18 Fatty Alcohol)⁴ | 2.84 | 2.84 | 2.84 | 2.84 | 2.84 |
| wt % (active) | | | | | |
| Sodium Benzoate⁵ wt % (active) | 0.25 | 0.25 | 0.25 | 0.25 | 0.25 |
| Decylene Glycol⁶ wt % (active) | 0.4 | 0.4 | 0.4 | 0.4 | 0.4 |
| Linoleamidopropyl Dimethylamine Dimer | 0.75 | 0.50 | 0.50 | 1.00 | 0.75 |
| Dilinoleate⁹ wt % (active) | | | | | |
| Diheptyl Succinate¹⁰ wt % (active) | 0.11 | 0.11 | 0.22 | 0.11 | 0.11 |
| Capryloyl Glycerin/Sebacic Acid Copolymer¹¹ | 0.14 | 0.14 | 0.28 | 0.14 | 0.14 |
| wt % (active) | | | | | |
| Avocado Oil wt % | | 1.00 | 1.00 | 1.00 | 1.00 |
| Perfume | 0.60 | 0.60 | 0.60 | 0.60 | 0.60 |
| Distilled Water | Q.S. | Q.S. | Q.S. | Q.S. | Q.S. |
| Adjust pH w Citric acid to pH = 3.5-4.5 | | | | | |

TABLE 4
Compositional Examples

| | Ex. 11 | Ex. 12 | Ex. 13 | Ex. 14 | Ex. 15 | Ex. 16 |
|---|---|---|---|---|---|---|
| Sheer stress (Pa) @950 1/s | 255 | 315 | 316 | 325 | 329 | 347 |
| DSC peak (melting temp) | 1 peak | 1 peak | 1 peak | 1 peak | 1 peak | 1 peak |
| GN d-spacing (nm) | | 23.2 | 23.2 | 23.0 | 23.0 | 22.8 |
| Behenamidopropyl Dimethylamine | 3.74 | 3.74 | 3.74 | 3.74 | 3.74 | 3.74 |
| (BAPDMA)¹ wt % (active) | | | | | | |
| L-Glutamic Acid² wt % (active) | 1.35 | 1.35 | 1.35 | 1.35 | 1.35 | 1.35 |
| Cetyl Alcohol (C16 Fatty alcohol)³ | 3.83 | 3.83 | 3.83 | 3.83 | 3.83 | 3.83 |
| wt % (active) | | | | | | |
| Stearyl Alcohol (C18 Fatty | 2.84 | 2.84 | 2.84 | 2.84 | 2.84 | 2.84 |
| Alcohol)⁴ wt % (active) | | | | | | |
| Sodium Benzoate⁵ wt % (active) | 0.25 | 0.25 | 0.25 | 0.25 | 0.25 | 0.25 |
| Decylene Glycol⁶ wt % (active) | 0.4 | 0.4 | 0.4 | 0.4 | 0.4 | 0.4 |
| Linoleamidopropyl Dimethylamine | 0.50 | 0.75 | 0.75 | 0.75 | 0.75 | 0.75 |
| Dimer Dilinoleate⁹ wt % (active) | | | | | | |
| Diheptyl Succinate¹⁰ wt % (active) | 0.11 | 0.11 | 0.11 | 0.11 | 0.11 | 0.11 |
| Capryloyl Glycerin/Sebacic Acid | 0.14 | 0.14 | 0.14 | 0.14 | 0.14 | 0.14 |
| Copolymer¹¹ wt % (active) | | | | | | |
| Safflower Oil wt % | 1.00 | 0.25 | 0.50 | 1.00 | 2.00 | 5.00 |
| Perfume | 0.60 | 0.60 | 0.60 | 0.60 | 0.60 | 0.60 |
| Distilled Water | Q.S. | Q.S. | Q.S. | Q.S. | Q.S. | Q.S. |
| Adjust pH w Citric acid to | | | | | | |
| pH = 3.5-4.5 | | | | | | |

TABLE 5
Compositional Examples

| | Ex. 17 | Ex. 18 | Ex. 19 | Ex. 20 | Ex. 21 |
|---|---|---|---|---|---|
| Sheer stress (Pa) @950 1/s | 325 | 324 | 327 | 333 | 358 |
| DSC peak (melting temp) | 1 peak | 1 peak | 1 peak | 1 peak | 1 peak |
| GN d-spacing (nm) | 23.0 | 23.0 | 23.0 | 22.8 | 22.6 |
| Behenamidopropyl Dimethylamine (BAPDMA)¹ | 3.74 | 3.74 | 3.74 | 3.74 | 3.74 |
| wt % (active) | | | | | |
| L-Glutamic Acid² wt % (active) | 1.35 | 1.35 | 1.35 | 1.35 | 1.35 |
| Cetyl Alcohol (C16 Fatty alcohol)³ | 3.83 | 3.83 | 3.83 | 3.83 | 3.83 |
| wt % (active) | | | | | |
| Stearyl Alcohol (C18 Fatty Alcohol)⁴ | 2.84 | 2.84 | 2.84 | 2.84 | 2.84 |
| wt % (active) | | | | | |
| Sodium Benzoate⁵ wt % (active) | 0.25 | 0.25 | 0.25 | 0.25 | 0.25 |
| Decylene Glycol⁶ wt % (active) | 0.4 | 0.4 | 0.4 | 0.4 | 0.4 |
| Linoleamidopropyl Dimethylamine Dimer | 0.75 | 0.75 | 0.75 | 0.75 | 0.75 |
| Dilinoleate⁹ wt % (active) | | | | | |
| Diheptyl Succinate¹⁰ wt % (active) | 0.11 | 0.11 | 0.11 | 0.11 | 0.11 |
| Capryloyl Glycerin/Sebacic Acid Copolymer¹¹ | 0.14 | 0.14 | 0.14 | 0.14 | 0.14 |
| wt % (active) | | | | | |
| Moringa Oil wt % | 0.25 | 0.50 | 1.00 | 2.00 | 5.00 |
| Perfume | 0.60 | 0.60 | 0.60 | 0.60 | 0.60 |
| Distilled Water | Q.S. | Q.S. | Q.S. | Q.S. | Q.S. |
| Adjust pH w Citric acid to pH = 3.5-4.5 | | | | | |

TABLE 6
Compositional Examples

| | Ex. 22 | Ex. 23 | Ex. 24 | Ex. 25 | Ex. 26 |
|---|---|---|---|---|---|
| Sheer stress (Pa) @950 1/s | 327 | 325 | 327 | 325 | 332 |
| DSC peak (melting temp) | 1 peak | 1 peak | 1 peak | 1 peak | 1 peak |
| GN d-spacing (nm) | | | | | 22.8 |
| Behenamidopropyl Dimethylamine (BAPDMA)¹ | 3.74 | 3.74 | 3.74 | 3.74 | 3.74 |
| wt % (active) | | | | | |
| L-Glutamic Acid² wt % (active) | 1.35 | 1.35 | 1.35 | 1.35 | 1.35 |
| Cetyl Alcohol (C16 Fatty alcohol)³ | 3.83 | 3.83 | 3.83 | 3.83 | 3.83 |
| wt % (active) | | | | | |
| Stearyl Alcohol (C18 Fatty Alcohol)⁴ | 2.84 | 2.84 | 2.84 | 2.84 | 2.84 |
| wt % (active) | | | | | |
| Sodium Benzoate⁵ wt % (active) | 0.25 | 0.25 | 0.25 | 0.25 | 0.25 |
| Decylene Glycol⁶ wt % (active) | 0.4 | 0.4 | 0.4 | 0.4 | 0.4 |
| Linoleamidopropyl Dimethylamine Dimer | 0.75 | 0.75 | 0.75 | 0.75 | 0.75 |
| Dilinoleate⁹ wt % (active) | | | | | |
| Diheptyl Succinate¹⁰ wt % (active) | 0.11 | 0.11 | 0.11 | 0.11 | 0.11 |
| Capryloyl Glycerin/Sebacic Acid Copolymer¹¹ | 0.14 | 0.14 | 0.14 | 0.14 | 0.14 |
| wt % (active) | | | | | |
| Camellia Oil wt % | 0.50 | | | | |
| Jojoba Oil wt % | | 0.50 | | | |
| Evening Primrose Oil wt % | | | 0.50 | | |
| Hemp Oil wt % | | | | 0.50 | |
| Bis-Aminopropyl Dimethicone wt % | | | | | 1 |
| Perfume | 0.60 | 0.60 | 0.60 | 0.60 | 0.60 |
| Distilled Water | Q.S. | Q.S. | Q.S. | Q.S. | Q.S. |
| Adjust pH w Citric acid to pH = 3.5-4.5 | | | | | |

TABLE 7
Comparative Compositional Examples

| | Comp. | Comp. | Comp. | Comp. | Comp. | Comp. | Comp. |
|---|---|---|---|---|---|---|---|
| | Ex. 5 | Ex. 6 | Ex. 7 | Ex. 8 | Ex. 9 | Ex. 10 | Ex. 11 |
| Sheer stress (Pa) @950 1/s | 338 | 381 | 368 | 391 | 324 | 366 | 391 |
| DSC peak (melting temp) | 1 peak | 1 peak | 1 peak | 1 peak | 1 peak | 1 peak | 1 peak |
| GN d-spacing (nm) | 23.0 | 23.0 | 22.6 | 22.6 | 23.2 | 22.6 | 22.0 |
| Behenamidopropyl Dimethylamine | 3.74 | 3.74 | 3.74 | 3.74 | 3.74 | 3.74 | 3.74 |
| (BAPDMA)¹ wt % (active) | | | | | | | |
| L-Glutamic Acid² wt % (active) | 1.35 | 1.35 | 1.35 | 1.35 | 1.35 | 1.35 | 1.35 |
| Cetyl Alcohol (C16 Fatty alcohol)³ | 3.83 | 3.83 | 3.83 | 3.83 | 3.83 | 3.83 | 3.83 |
| wt % (active) | | | | | | | |
| Stearyl Alcohol (C18 Fatty | 2.84 | 2.84 | 2.84 | 2.84 | 2.84 | 2.84 | 2.84 |
| Alcohol)⁴ wt % (active) | | | | | | | |
| Sodium Benzoate⁵ wt % (active) | 0.25 | 0.25 | 0.25 | 0.25 | 0.25 | 0.25 | 0.25 |
| Caprylyl Glycol⁷ wt % (active) | 0.2 | 0.2 | 0.2 | 0.2 | 0.2 | 0.2 | 0.2 |
| 1,2 Hexandiol⁸ wt % (active) | 0.2 | 0.2 | 0.2 | 0.2 | 0.2 | 0.2 | 0.2 |
| Safflower Oil wt % | | 1 | 2 | 5 | | | |
| Moringa Oil wt % | | | | | 1 | 2 | 5 |
| Perfume | 0.60 | 0.60 | 0.60 | 0.60 | 0.60 | 0.60 | 0.60 |
| Distilled Water | Q.S. | Q.S. | Q.S. | Q.S. | Q.S. | Q.S. | Q.S. |
| Adjust pH w Citric acid to | | | | | | | |
| pH = 3.5-4.5 | | | | | | | |

TABLE 8
Comparative Compositional Examples of Non-Silicone Hair Conditioning Agents

| | Comp. | Comp. | Comp. | Comp. | Comp. | Comp. | Comp. |
|---|---|---|---|---|---|---|---|
| | Ex. 12 | Ex. 13 | Ex. 14 | Ex. 15 | Ex. 16 | Ex. 17 | Ex. 18 |
| Viscosity cps @ 2 s⁻¹ | 8524 | 5434 | 22 | 23 | 10247 | 6929 | 5978 |
| Viscosity cps @950 s⁻¹ | 7326 | 4898 | 19 | 20 | 6167 | 5809 | 5177 |
| Linoleamidopropyl Dimethylamine | 100 | | | | 50 | 10 | 90 |
| Dimer Dilinoleate⁹ wt % (active) | | | | | | | |
| Diheptyl Succinate¹⁰ wt % (active) | | | 98 | | | | 4.5 |
| Diisooctyl Succinate¹² wt % (active) | | | | 100 | | | |
| Capryloyl Glycerin/Sebacic Acid | | 100 | 2 | | 50 | 90 | 5.5 |
| Copolymer¹¹ wt % (active) | | | | | | | |

TABLE 9
Compositional Examples of Non-Silicone Hair Conditioning Agents

| | Ex. 27 | Ex. 28 | Ex. 29 | Ex. 30 | Ex. 31 | Ex. 32 | Ex. 33 |
|---|---|---|---|---|---|---|---|
| Viscosity cps @ 2 s⁻¹ | 4739 | 3457 | 4463 | 2826 | 5234 | 505 | 4291 |
| Viscosity cps @950 s⁻¹ | 3848 | 3109 | 3372 | 2158 | 3715 | 474 | 3327 |
| Linoleamidopropyl Dimethylamine | 80 | 10 | 60 | 60 | 70 | 10 | 75 |
| Dimer Dilinoleate⁹ wt % (active) | | | | | | | |
| Diheptyl Succinate¹⁰ wt % (active) | 9 | 9.8 | 9.8 | 18 | | 40.5 | 11.25 |
| Diisooctyl Succinate¹² wt % (active) | | | | | 10 | | |
| Capryloyl Glycerin/Sebacic Acid | 11 | 80.2 | 30.2 | 22 | 20 | 49.5 | 13.75 |
| Copolymer¹¹ wt % (active) | | | | | | | |

TABLE 10
Compositional Examples of Non-Silicone Hair Conditioning Agents

| | Ex. 34 | Ex. 35 | Ex. 36 | Ex. 37 | Ex. 38 | Ex. 39 | Ex. 40 |
|---|---|---|---|---|---|---|---|
| Viscosity cps @ 2 s⁻¹ | 756 | 2693 | 1310 | 2085 | 4781 | 2826 | 3794 |
| Viscosity cps @950 s⁻¹ | 641 | 1604 | 864 | 1170 | 3111 | 2158 | 2804 |
| Linoleamidopropyl Dimethylamine | 20 | 50 | 30 | 40 | 45 | 60 | 70 |
| Dimer Dilinoleate⁹ wt % (active) | | | | | | | |
| Diheptyl Succinate¹⁰ wt % (active) | 36 | 22.5 | 31.5 | 27 | 9.8 | 18 | 13.5 |
| Capryloyl Glycerin/Sebacic Acid | 44 | 27.5 | 38.5 | 33 | 45.2 | 22 | 16.5 |
| Copolymer¹¹ wt % (active) | | | | | | | |

TABLE 11
Comparative and Compositional Examples

| | Comp. | | | | | | |
|---|---|---|---|---|---|---|---|
| | Ex. 19 | Ex. 41 | Ex. 42 | Ex. 43 | Ex. 44 | Ex. 45 | Ex. 46 |
| Uniformity of particles of hair | Not | uniform | uniform | uniform | uniform | uniform | uniform |
| conditioning agent in GN | uniform | | | | | | |
| Particle sizes (mm) of hair | >300 | 1-10 | 1-15 | 1-10 | 1-5 | 1-10 | 1-5 |
| conditioning agent in GN | | | | | | | |
| Viscosity cps @ 2 s⁻¹ of | 8524 | 505 | 3457 | 756 | 4739 | 4291 | 5234 |
| hair conditioning agent | | | | | | | |
| composition | | | | | | | |
| Viscosity cps @950 s⁻¹ of | 7326 | 474 | 3109 | 641 | 3848 | 3327 | 3715 |
| hair conditioning agent | | | | | | | |
| composition | | | | | | | |
| Sheer stress (Pa) @950 1/s | N/A | 407 | 430 | 402 | 330 | 344 | 385 |
| of conditioner composition | | | | | | | |
| Behenamidopropyl | 3.74 | 3.74 | 3.74 | 3.74 | 3.74 | 3.74 | 3.74 |
| Dimethylamine | | | | | | | |
| (BAPDMA)¹ wt % (active) | | | | | | | |
| L-Glutamic Acid² wt % | 1.35 | 1.35 | 1.35 | 1.35 | 1.35 | 1.35 | 1.35 |
| (active) | | | | | | | |
| Cetyl Alcohol (C16 Fatty | 3.83 | 3.83 | 3.83 | 3.83 | 3.83 | 3.83 | 3.83 |
| alcohol)³ wt % (active) | | | | | | | |
| Stearyl Alcohol (C18 Fatty | 2.84 | 2.84 | 2.84 | 2.84 | 2.84 | 2.84 | 2.84 |
| Alcohol)⁴ wt % (active) | | | | | | | |
| Sodium Benzoate⁵ wt % | 0.25 | 0.25 | 0.25 | 0.25 | 0.25 | 0.25 | 0.25 |
| (active) | | | | | | | |
| Caprylyl Glycol⁷ wt % | 0.2 | 0.2 | 0.2 | 0.2 | 0.2 | 0.2 | 0.2 |
| (active) | | | | | | | |
| 1,2 Hexandiol⁸ wt % (active) | 0.2 | 0.2 | 0.2 | 0.2 | 0.2 | 0.2 | 0.2 |
| Linoleamidopropyl | 3.00 | 0.3 | 0.3 | 0.6 | 2.4 | 2.25 | 2.1 |
| Dimethylamine Dimer | | | | | | | |
| Dilinoleate⁹ wt % (active) | | | | | | | |
| Diheptyl Succinate¹⁰ wt % | | 1.215 | 0.294 | 1.08 | 0.27 | 0.3375 | |
| (active) | | | | | | | |
| Diisooctyl Succinate¹² wt % | | | | | | | 0.3 |
| (active) | | | | | | | |
| Capryloyl Glycerin/Sebacic | | 1.485 | 2.406 | 1.32 | 0.33 | 0.4125 | 0.6 |
| Acid Copolymer¹¹ wt % | | | | | | | |
| (active) | | | | | | | |
| Distilled Water | Q.S. | Q.S. | Q.S. | Q.S. | Q.S. | Q.S. | Q.S. |
| Adjust pH w Citric aicd to | | | | | | | |
| pH = 3.5-4.5 | | | | | | | |

¹Behenamidopropyl Dimethylamine (BAPDMA) (Incromine ™ BD), available from Croda ®
²L-Glutamic Acid, available from Ajinomoto ®
³Cetyl alcohol, 95 wt % active level available from Procter & Gamble ®
⁴Stearyl alcohol, 97 wt % active level, available from Procter & Gamble ®
⁵Sodium Benzoate, available from Kalama ®
⁶Decylene Glycol (SymClariol ®), available from Symrise ®
⁷Caprylyl Glycol, 50 wt % active level, available from Symrise ® under tradename of Hydrolite ® CG
⁸1,2 Hexandiol, 50 wt % active level, available from Symrise ® under tradename of Hydrolite ® CG
⁹Linoleamidopropyl Dimethylamine Dimer Dilinoleate (Necon ™ LO-80), available from Alzo International ®
¹⁰Diheptyl Succinate, 45 wt % active level, available from Inolex ® under tradename of LexFeel ™ N350 MB; 98 wt % active level, available from Inolex ® under tradename of LexFeel ™ N5 MB
¹¹Capryloyl Glycerin/Sebacic Acid Copolymer, 55 wt % active level, available from Inolex ® under tradename of LexFeel ™ N350 MB.
¹²Diheptyl Succinate, 100 wt % active level, available from Inolex ® under tradename of Sustoleo ™ DCS.

The ratios, viscosities, and particle size of the non-silicone hair conditioning materials may be as follows:

Where (b) is a dicarboxylic acid amine salt; (c) is a diester; and (d) is a glycerin ester copolymer, the ratio of (b):(c) may be from about 10:1 to about 1:10, and in some embodiments, from about 9:1 to about 1:7. The ratio of (d):(c) may be from about 10:1 to about 1:10, and in some embodiments, from about 9:1 to about 1:7. The ratio of (b):(d) may be from about 20:1 to about 1:20, in some embodiments from about 10:1 to about 1:10. In Table 8, Comparative Example 18, the ratio of b:c is 20 and is outside of the inventive ratio of 10:1 to 1:10. The ratios of the materials in Tables 9 and 10 are within the inventive ranges.

The viscosity of a composition of the non-silicone hair conditioning materials (a dicarboxylic acid amine salt+a diester+a glycerin ester copolymer) may be less than 5000 @ 950 1/s (high shear rate), and in some embodiments less than 4500 @ 950 1/s. In Table 8, Comparative Example 18, the viscosity of the three materials is greater than 5000 @ 950 1/s, while for the inventive combinations in Tables 9 and 10 the viscosity is less than 5000 @ 950 1/s.

The particle size of the non-silicone hair conditioning materials (a dicarboxylic acid amine salt+a diester+a glycerin ester copolymer), when suspended uniformly in an L-basal lamellar gel network, may be less than 100 microns, and in some embodiments less than 50 microns, as measured by the Optical Microscope Image Method described herein. Table 11 shows the particle size of Comparative Example 19 (higher than 100 microns) versus the particle size of inventive examples 41-46 (less than 100 microns).

### Process of Making

It is preferred to prepare the composition by the following method:

Water is typically heated to at least about 70° C., preferably between about 80° C. and about 90° C. The cationic surfactant and the high melting point fatty compound are combined with the water to form a mixture. The temperature of the mixture is preferably maintained at a temperature higher than both the melting temperature of the cationic surfactant and the melting temperature of the high melting point fatty compound, and the entire mixture is homogenized. After mixing until no solids are observed, the mixture is gradually cooled (e.g., at a rate of from about 1° C./minute to about 5° C./minute) to a temperature below 60° C., preferably less than about 55° C. During this gradual cooling process, a significant viscosity increase is observed at between about 55° C. and about 75° C. This indicates the formation of gel matrix. The high molecular weight water-soluble cationic polymer can be added to the mixture with agitation at about 55° C., or prior to the cooling down. Additional components are then combined with the gel matrix and cooled to room temperature.

The non-silicone hair conditioning agent compositions were incorporated into the conditioner compositions after the L-basal lamellar gel network formed.

### COMBINATIONS

A. A hair conditioner composition comprising:
   (a) a L-basal lamellar gel network;
   (b) from about 0.01 wt % to about 5 wt % of a dicarboxylic acid amine salt;
   (c) from about 0.01 wt % to about 5 wt % of a diester;
   (d) from about 0.01 wt % to about 5 wt % of a glycerin ester copolymer; wherein the composition has a shear stress from about 40 Pa to about 800 Pa @ 950 1/s.
B. The composition according to paragraph A, further comprising from about 0.1 wt % to about 15 wt % of a natural oil or wax selected from the group consisting of natural oils from plants and vegetables, coconut oil, corn oil, cottonseed oil, canola oil, olive oil, palm oil, peanut oil, safflower oil, sesame oil, soybean oil, sunflower oil, jojoba oil, shea butter, cocoa butter, pequi oil, argan oil, almond oil, apricot oil, rice bran oil, safflower oil, hemp seed oil, avocado oil, grapeseed oil, evening primrose oil, camelia oil, moringa oil, meadowfoam oil, crambe oil, castor oil, candelilla wax, rice bran wax, sunflower wax, beeswax, bayberry wax, orange wax, carnauba wax, and mixtures thereof.
C. The composition according to paragraphs A and B, wherein the L-basal lamellar gel network comprises (a) an aqueous carrier; (b) from about 0.1 wt % to about 20 wt % of a cationic surfactant; and (c) from about 0.1 wt % to about 20 wt % of a fatty alcohol.
D. The composition according to paragraphs A to C, wherein the L-basal lamellar gel network comprises d-spacing of from about 5 nm to about 50 nm, as measured according to the d-spacing (L-basal spacing) of Lamella Gel Network Test Method.
E. The composition according to paragraphs A to D, wherein the dicarboxylic acid amine salt is a reaction product of a dicarboxylic acid and an amine;
   a. wherein the dicarboxylic acid is selected from C36 aliphatic alkyl dicarboxylic acid, C36 monocycloaliphatic alkyl dicarboxylic acid, dilinoleic acid, and mixtures thereof;
   b. wherein the amine is selected from: mono-long alkyl amines; mono-long alkyl dimethylamine, mono-long alkyl amidoamines, mono-long alkyl amidopropyl dimethylamines, lauryldimethylamine, hexadecyldimethylamine, linoleamidopropyldimethylamine, behenamidopropyldimethylamine, behenamidopropyldiethylamine, behenamidoethyldiethylamine, behenamidoethyldimethylamine, brassicamidopropyldimethylamine, brassicamidopropyldiethylamine, brassicamidoethyldiethylamine, brassicamidoethyldimethylamine amines, stearamidopropyldimethylamine, stearamidopropyldiethylamine, stearamidoethyldiethylamine, stearamidoethyldimethylamine, palmitamidopropyldimethylamine, palmitamidopropyldiethyl amine, palmitamidoethyldiethylamine, palmitamidoethyldimethylamine, arachidamidopropyldimethylamine, arachidamidopropyldiethylamine, arachidamidoethyldiethylamine, arachidamidoethyldimethylamine, and/or diethylaminoethylstearamide.
F. The composition according to paragraphs A to E, wherein the dicarboxylic acid amine salt is selected from linoleamidopropyldimethylamine dimer dilinoleate, behenamidopropyldimethylamine dimer dilinoleate, lauryldimethylamine dimer dilinoleate, hexadecyldimethylamine dimer clilinoleate, and mixtures thereof.
G. The composition according to paragraphs A to F, wherein the diester has the formula (VII):

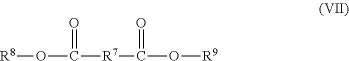

wherein R⁷ is a C₁ to C₃₆ straight, cyclic, or branch drained, saturated or unsaturated hydrocarbon group; wherein R⁸ and R⁹ are C₁ to C₂₂ straight, cyclic, or branch chained, saturated or unsaturated hydrocarbon groups.
H. The composition according to paragraphs A to G, wherein the diester is selected from the group consisting of diheptyl succinate, dipenyl succinate, didecyl succinate, dicapryl succinate, diheptyl suberate, dipenyl suberate, didecyl suberate, diheptyl sebacate, dipenyl sebacate, didecyl sebacate, diheptyl oxalate, dipenyl oxalate, didecyl oxalate, dioctyl adipate, ditetradecyl sebacate, bis(2thyl-1-hexyl) adipate, and mixtures thereof.
I. The composition according to paragraphs A to H, wherein the viscosity of the diester is less than about 100 cps.
J. The composition according to paragraphs A to I, wherein the glycerin ester copolymer is a reaction product of (a) a polyfunctional alcohol, (b) a polyfunctional carboxylic acid, and (c) a monocarboxylic acid.
K. The composition according to paragraph J, wherein the polyfunctional alcohol is selected from the group consisting of glycerol, pentaerythritol, dipentaerythritol, tripentaerythritol, trimethylolpropane, neopentyl glycol, propylene glycol, 1,3-butylene glycol, 2-methyl-1,3-propanediol, dipropylene glycol, ethylene glycol, cyclohexanedimethanol, butyl ethyl propanediol, and derivatives and combinations thereof.
L. The composition according to paragraphs J and K, wherein the polyfunctional carboxylic acid is selected from the group consisting of carbonic acid, hexanedioic acid, dimer acid, azelaic acid, sebacic acid, dodecanedioic acid, glutaric acid, succinic acid, phthalic acid, isophthalic acid, terephthalic acid, 2,6-naphthalene dicarboxylic acid, and derivatives and combinations thereof.
M. The composition according to paragraphs J to L, wherein the monocarboxylic acid is selected from the group consisting of isobutyric acid, benzoic nonanoic acid, 3,5,5-trimethylhexanoic acid, isononanoic acid, decanoic acid, isooctadecanoic acid, dodecanoic acid, 2-methyl butyric acid, isopentanoic acid, pentanoic acid, 2-methyl pentanoic acid, 2-methyl hexanoic acid, isooctanoic acid, undecylinic acid, isolauric acid, isopalmitic acid, isostearic acid, behenic acid, and derivatives and combinations thereof.
N. The composition according to paragraphs A to M, wherein the glycerin ester copolymer is a reaction product of (a) glycerin, (b) sebacic acid, and (c) caprylic acid.
O. The composition according to paragraphs A to N, wherein the viscosity of the glycerin ester copolymer is from about 200 to about 5000 cps.
P. The composition according to paragraphs A to O, wherein the hydroxyl value of the glycerin ester copolymer is from about 40 to about 300 mg KOH/g, as measured by AOCS (American Oil Chemists Society, Champaign, Ill., United States of America) official method Cd 13-60.
Q. A method of conditioning hair, using the hair conditioner composition according to paragraphs A to P.

What is claimed is:

1. A hair conditioner composition comprising:
(a) a L-basal lamellar gel network;
(b) from about 0.01 wt % to about 5 wt % of a dicarboxylic acid amine salt;
(c) from about 0.01 wt % to about 5 wt % of a diester;
(d) from about 0.01 wt % to about 5 wt % of a glycerin ester copolymer;
wherein the composition has a shear stress from about 40 Pa to about 800 Pa @ 950 1/s.

1. The composition of claim 1, further comprising from about 0.1 wt % to about 15 wt % of a natural oil or wax selected from the group consisting of natural oils from plants and vegetables, coconut oil, corn oil, cottonseed oil, canola oil, olive oil, palm oil, peanut oil, safflower oil, sesame oil, soybean oil, sunflower oil, jojoba oil, shea butter, cocoa butter, pequi oil, argan oil, almond oil, apricot oil, rice bran oil, safflower oil, hemp seed oil, avocado oil, grapeseed oil, evening primrose oil, camelia oil, moringa oil, meadowfoam oil, crambe oil, castor oil, candelilla wax, rice bran wax, sunflower wax, beeswax, bayberry wax, orange wax, carnauba wax, and mixtures thereof.

2. The composition of claim 1, wherein the L-basal lamellar gel network comprises (a) an aqueous carrier; (b) from about 0.1 wt % to about 20 wt % of a cationic surfactant; and (c) from about 0.1 wt % to about 20 wt % of a fatty alcohol.

3. The composition of claim 1, wherein the L-basal lamellar gel network comprises d-spacing of from about 5 nm to about 50 nm, as measured according to the d-spacing (L -basal spacing) of Lamella Gel Network Test Method.

4. The composition of claim 1, wherein the dicarboxylic acid amine salt is a reaction product of a dicarboxylic acid and an amine;
a. wherein the dicarboxylic acid is selected from C36 aliphatic alkyl dicarboxylic acid, C36 monocycloaliphatic alkyl dicarboxylic acid, dilinoleic acid, and mixtures thereof;
b. wherein the amine is selected from: mono-long alkyl amines; mono-long alkyl dimethylamine, mono-long alkyl amidoamines, mono-long alkyl amidopropyl dimethylamines, lauryldimethylamine, hexadecyldimethylamine, linoleamidopropyldimethylamine, behenamidopropyldimethylamine, behenamidopropyldiethylamine, behenamidoethyldiethylamine, behenamidoethyldimethylamine, brassicamidopropyldimethylamine, brassicamidopropyldiethylamine, brassicamidoethyldiethylamine, brassicamidoethyldimethylamine amines, stearamidopropyldimethylamine, stearamidopropyldiethylamine, stearamidoethyldiethylamine, stearamidoethyldimethylamine, palmitamidopropyldimethylamine, palmitamidopropyldiethyl amine, palmitamidoethyldiethylamine, palmitamidoethyldimethylamine, arachidamidopropyldimethylamine, arachidamidopropyldiethylamine, arachidamidoethyldiethylamine, arachidamidoethyldimethylamine, and/or diethylaminoethylstearamide.

5. The composition of claim 1, wherein the dicarboxylic acid amine salt is selected from linoleamidopropyldimethylamine climes dilinoleate, behenamidopropyldimethylamine dimer dilinoleate, lauryldimethylamine dimer dilinoleate, hexadecyldimethylamine dimer dilinoleate, and mixtures thereof.

6. The composition of claim 1, wherein the diester has the formula (VII): 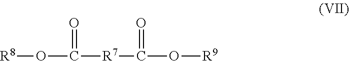
wherein R⁷ is a C₁ to C₃₆ straight, cyclic, or branch chained, saturated or unsaturated hydrocarbon group; wherein R⁸ and R⁹ are C₁ to C₂₂ straight, cyclic or branch chained, saturated or unsaturated hydrocarbon groups.

7. The composition of claim 1, wherein the diester is selected from the group consisting of diheptyl succinate, dipenyl succinate, didecyl succinate, dicapryl succinate, diheptyl suberate, dipenyl suberate, didecyl suberate, diheptyl sebacate, dipenyl sebacate, didecyl sebacate, diheptyl oxalate, dipenyl oxalate, didecyl oxalate, dioctyl adipate, ditetradecyl sebacate, bis(2thyl-1-hexyl) adipate, and mixtures thereof.

8. The composition of claim 1, wherein the viscosity of the diester is less than about 100 cps.

9. The composition of claim 1, wherein the glycerin ester copolymer is a reaction product of (a) a polyfunctional alcohol, (b) a polyfunctional carboxylic acid, and (c) a monocarboxylic acid.

10. The composition of claim 10, wherein the polyfunctional alcohol is selected from the group consisting of glycerol, pentaerythritol, dipentaerythritol, tripentaerythritol, trimethylolpropane, neopentyl glycol, propylene glycol, 1,3-butylene glycol, 2-methyl-1,3-propanediol, dipropylene glycol, ethylene glycol, cyclohexanedimethanol, butyl ethyl propanediol, and derivatives and combinations thereof.

11. The composition of claim 10, wherein the polyfunctional carboxylic acid is selected from the group consisting of carbonic acid, hexanedioic acid, dimer acid, azelaic acid, sebacic acid, dodecanedioic acid, glutaric acid, succinic acid, phthalic acid, isophthalic acid, terephthalic acid, 2,6-naphthalene dicarboxylic acid, and derivatives and combinations thereof.

12. The composition of claim 10, wherein the monocarboxylic acid is selected from the group consisting of isobutyric acid, benzoic nonanoic acid, 3,5,5-trimethylhexanoic acid, isononanoic acid, decanoic acid, isooctadecanoic acid, dodecanoic acid, 2-methyl butyric acid, isopentanoic acid, pentanoic acid, 2-methyl pentanoic acid, 2-methyl hexanoic acid, isooctanoic acid, undecylinic acid, isolauric acid, isopalmitic acid, isostearic acid, behenic acid, and derivatives and combinations thereof.

13. The composition of claim 1, wherein the glycerin ester copolymer is a reaction product of (a) glycerin, (b) sebacic acid, and (c) caprylic acid.

14. The composition of claim 1, wherein the viscosity of the glycerin ester copolymer is from about 200 to about 5000 cps.

15. The composition of claim 1, wherein the hydroxyl value of the glycerin ester copolymer is from about 40 to about 300 mg KOH/g, as measured by AOCS (American Oil Chemists Society, Champaign, Illinois, United States of America) official method Cd 13-60.

16. A method of conditioning hair, using the hair conditioner composition of claim 1.

17. The composition of claim 1, wherein the viscosity of the mixture of (b), (c), and (d) is less than 5000 @ 950 1/s.

18. The composition of claim 1, wherein the ratio of (b):(c) is from about 10:1 to about 1:10; the ratio of (d):(c) is from about 10:1 to about 1:10; and the ratio of (b):(d) is from about 20:1 to about 1:20.

19. The composition of claim 1, wherein the particle size of the mixture of (b), (c), and (d), when suspended in the L-basal lamellar gel network, is less than 100 microns.
